(19) United States
(12) Patent Application Publication
(10) Pub. No.: US 2010/0106144 A1
(43) Pub. Date: Apr. 29, 2010

(54) PUNCTURE DEVICE AND METHOD FOR CONTROLLING SAME

(76) Inventors: Keisuke Matsumura, Ehime (JP); Kiyohiro Horikawa, Ehime (JP)

Correspondence Address: WENDEROTH, LIND & PONACK L.L.P., 1030 15th Street, N.W., Suite 400 East, Washington, DC, 20005-1503, US

(21) Appl. No.: 12/582,278

(22) Filed: Oct. 20, 2009

(30) Foreign Application Priority Data
Oct. 29, 2008 (JP) 2008-277967

(51) Int. Cl. A61B 18/20

(52) U.S. Cl. 606/10

(57) ABSTRACT

A puncture device 21 comprises capacitors 24 and 33 to which voltage is applied, a laser puncture unit 25 to which the output of these capacitors 24 and 33 is supplied, a trigger circuit 30 that applies a trigger voltage to the laser puncture unit 25, and a controller 35 that controls this trigger circuit 30. The puncture device 21 further comprises switches 29 and 32 that switch the connection of the capacitors 24 and 33. The switches 29 and 32 are controlled by the controller 35 so as to carry out a first puncture, which is performed with the capacitors 24 and 33 connected in parallel, and a second puncture, which is performed with the capacitors 24 and 33 connected in series.

### CROSS-REFERENCE TO RELATED APPLICATIONS

This application claims priority to Japanese Patent Application No. 2008-277967. The entire disclosure of Japanese Patent Application No. 2008-277967 is hereby incorporated herein by reference.

### BACKGROUND

1. Technical Field

The present invention relates to a puncture device that makes use of a laser, and to a method for controlling this device.

2. Description of the Prior Art

Many puncture methods that involve the use of a needle are currently being used as methods for collecting tiny amounts of blood from the skin, and many needle-type puncture devices are on the market. Nevertheless, because these products involve contact, there is the risk of infection. Accordingly, laser puncture devices have been developed which allow non-contact puncture to be performed.

A conventional laser puncture device will now be described. FIG. 13 is a block diagram of a conventional puncture device.

As shown in FIG. 13, a conventional puncture device 1 comprises a laser rod, a converging lens, a flash lamp, a lens barrel, a 600 μF capacitor, a battery 2, a booster circuit 4, a trigger circuit 7, a controller 8, and a puncture button 6c. The laser rod outputs a laser beam for puncturing the skin. The converging lens converges the laser beam outputted from the laser rod. The flash lamp excites the laser rod. The lens barrel converges the light outputted from the flash lamp onto the laser rod. The 600 μF capacitor applies voltage to the flash lamp. The battery 2 charges the capacitor. The booster circuit 4 is connected to the battery 2. The trigger circuit 7 applies a trigger voltage to the flash lamp. The controller 8 controls the trigger circuit 7 and the booster circuit 4. The puncture button 6c is connected to the controller 8.

The operation of the puncture device 1 constituted as above will be described through reference to FIGS. 13 and 14A to 14C.

In FIG. 14A, the vertical axis 10a is the level, and the horizontal axis 11 is the time. 12 (indicated by a solid line) is the voltage of the capacitor applied to a flash lamp 6a, and 13 (indicated by a dotted line) is the intensity of the light emitted from the flash lamp 6a.

First, when a power button 3a is pressed, the output of the battery 2 is boosted by the booster circuit 4, and charging of a capacitor 5 is commenced (not shown). The final charging voltage is set by the booster circuit 4 so as to be 300 V at a point 11a.

At a point 11b, when the puncture button 6c is pressed, a trigger voltage is generated by the trigger circuit 7, and a high voltage is applied to a trigger electrode 6b. When a high voltage is applied to the trigger electrode 6b, the flash lamp 6a emits light. When the flash lamp 6a emits light, some of the charge stored in the capacitor is consumed and the voltage 12 of the capacitor drops. The intensity of the light outputted from the flash lamp 6a peaks out and then decreases along with the voltage 12.

The laser rod is excited by the emission of the flash lamp, and a laser beam 6f is outputted from the laser rod. In FIG. 14B, the vertical axis 10b is the laser output, and 14 expresses the laser output 6f with respect to the emission of the flash lamp. The laser output 6f crosses an excitation threshold and stops once the optical intensity 13 decreases to a specific threshold 13a. This threshold 13a varies with the flash lamp, laser rod, and lens barrel converging efficiency, but in this Specification, we will assume the voltage of the capacitor to be 200 V. The laser beam outputted from the laser rod is converged by the converging lens and directed at the skin 15 (see FIG. 13), where the skin is punctured. Blood 16 seeps out from the punctured skin 15.

The puncture state will be described in detail through reference to the cross section of the puncture state shown in FIG. 15. In FIG. 15, when the laser beam 6f is directed at the skin 15 from the laser rod, the skin surface is eliminated by ablation. The skin at this point undergoes ablation with respect to the depth direction and spreading direction so that a wave spreads out from the irradiation site. The skin 15 is made up of the epidermis 15a through which no nerves or veins pass, and the dermis 15b, which is deeper than the epidermis 15a and in which nerves and veins are present. Therefore, to puncture the skin 15 and collect blood, the puncture must go down to the depth of the dermis 15b. This allows the blood 16 (see FIG. 13) to seep out. Here, if the laser beam irradiation time exceeds the thermal relaxation time, a heat conduction region 17a will be formed by the laser heat around the puncture hole 17.

Japanese Laid-Open Patent Application 2004-195245 is known, for example, as a prior art publication related to the invention of this application.

### SUMMARY

However, with this conventional puncture device 1, if the voltage decreases to 200 V, as shown beyond the point 11c in FIG. 14C, even though the flash lamp 6a is turned on, the laser beam 6f outputted from the laser rod 6d ends up stopping after the point 11c. Although the laser beam 6f is not outputted, the flash lamp 6a remains on, so energy efficiency suffers, which can be a particularly serious problem in portable devices that are battery driven.

This drop in energy efficiency will now be described in detail. In FIG. 14C, the vertical axis 10c is the electrical level, and 18 is the electrical energy with which the capacitor 5 is charged. At the point 11b at which the puncture button 6c is pressed, at first the capacitor 5 is charged with 27 J of energy, as shown below.

$$\begin{matrix}{E}_{1} = {C}_{1} {V}_{1}^{2} / 2 \\ = ( 600 µ F \times {300}^{2} ) / 2 \\ = 27 ( J )\end{matrix} \\ ( Where {C}_{1} = 600 µ F , and {V}_{1} = 300 V . ) \qquad ( 1 )$$

This 27 J of electrical energy is consumed by the light emission of the flash lamp 6a. At the point 11c when the laser beam 6f stops, the energy with which the capacitor 5 is charged is 12 J, as shown below.

$$\begin{matrix}{E}_{2} = {C}_{1} {V}_{2}^{2} / 2 \\ = ( 600 µ F \times {200}^{2} ) / 2 \\ = 12 ( J )\end{matrix} \\ ( Where {V}_{2} = 200 V . ) \qquad ( 2 )$$

Of the 27 J of energy with which the capacitor 5 is charged, the energy used for puncture is the difference thereof, or 15 J. The remaining 12 J of energy (roughly 40%) does not contribute to the emission of the laser beam 6f, and ends up being discarded as waste energy.

It is an object of the present invention to solve this problem, and to provide a puncture device with improved energy efficiency.

To achieve this object, the puncture device of the present invention comprises a plurality of capacitors and a switching circuit that switches the connection of these capacitors. The controller also controls the switching circuit so as to carry out a first puncture, which is performed with the capacitors connected in parallel, and a second puncture, which is performed with the capacitors connected in series at the same place as the first puncture. This allows the above-mentioned object to be achieved.

Specifically, the first puncture lowers the voltage with which the plurality of capacitors connected in parallel are charged. However, even though it drops to the level at which the emission of laser light stops, a voltage level at which the emission of the laser beam is possible can be ensured by connecting these capacitors in series. The second puncture can be performed in series at the same place as the first puncture. That is, a puncture can be performed and blood collected at a high energy efficiency, without wasting the energy with which the capacitors are charged.

Also, when a puncture is made with a laser, ablation occurs so that the puncture diameter becomes greater proportional to the puncture depth. Accordingly, since a puncture depth that goes all the way down to the dermis layer is obtained all at once, the puncture diameter inevitably increases, which causes more pain to the patient. Furthermore, since the irradiation time increases in a single irradiation, the heat of the laser may cause the tissue to coagulate, producing a hemostatic effect. Therefore, the patient has to make a larger hole in the skin surface, and this may further aggravate the patient's pain.

In contrast, when two or more punctures are made as in the present invention, the puncture diameter can be reduced by making a plurality of punctures at a shallower depth. Furthermore, since a single laser irradiation takes less time, the tissue of the skin surface is not affected by heat, and blood can be obtained with a smaller hole, so less pain is inflicted on the patient.

### BRIEF DESCRIPTION OF DRAWINGS

FIG. 1 is a block diagram of the laser puncture device in Embodiment 1 of the present invention;

FIG. 2 is a circuit diagram of the booster circuit of this device, and its surroundings;

FIGS. 3A to 3C are block diagrams of the operating state of this device, with FIG. 3A being a block diagram during the charging of the device, FIG. 3B a block diagram during the first puncture, and FIG. 3C a block diagram during the second puncture;

FIGS. 4A and 4B are cross sections of the state of puncture with the laser beam in this device, with FIG. 4A being a cross section of the puncture state after the first puncture, and FIG. 4B a cross section of the puncture state after the second puncture;

FIGS. 5A to 5D are timing charts of the puncture operation in this device, with FIG. 5A being a time chart of the optical intensity and voltage of the flash lamp, FIG. 5B a time chart of the laser output, FIG. 5C a time chart of the charge energy, and FIG. 5D a time chart of chop switch control;

FIG. 6 is a cross section of a blood testing device in Embodiment 2;

FIG. 7 is a cross section of the puncture component and its surrounding;

FIG. 8 is a cross section of a sensor;

FIG. 9 is a see-through plan view;

FIG. 10 is an oblique view;

FIG. 11 is a block diagram of the electrical circuit and its surroundings;

FIG. 12 is a flowchart of a blood testing method;

FIG. 13 is a block diagram of a conventional laser puncture device;

FIGS. 14A to 14C are timing charts of the puncture operation in this conventional device, with FIG. 14A being a time chart of the optical intensity and voltage of the flash lamp, FIG. 14B a time chart of the laser output, and FIG. 14C a time chart of the charge energy; and

FIG. 15 is a cross section of the state of puncture with the laser beam in this device.

### DETAILED DESCRIPTION OF THE PREFERRED EMBODIMENTS

### Embodiment 1

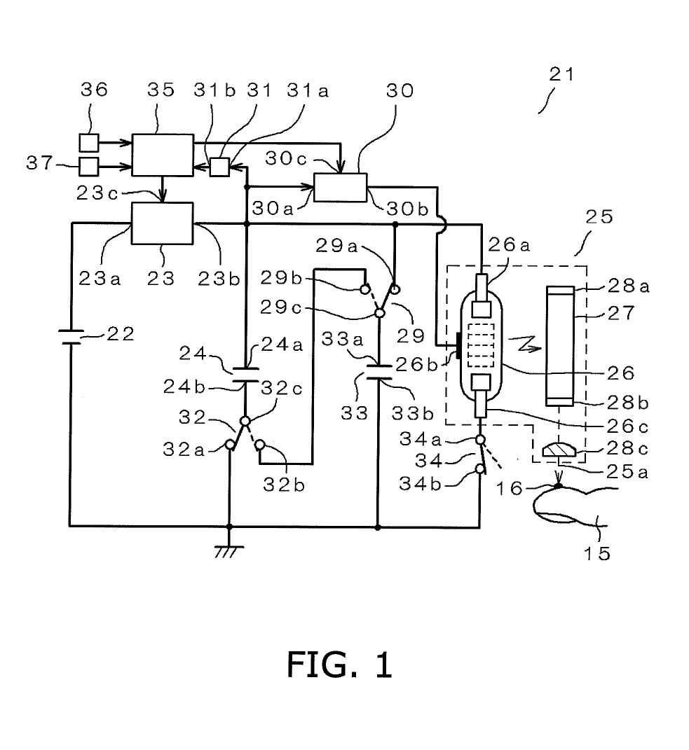

An embodiment of the present invention will now be described through reference to the drawings. FIG. 1 is a block diagram of a puncture device 21 in Embodiment 1. In FIG. 1, 22 is a lithium ion battery (an example of a power supply) that outputs a voltage of 3.7 V.

The negative side of the battery 22 is connected to ground, while the positive side is connected to an input 23a of a booster circuit 23. Either a primary cell or a secondary cell can be used as the battery 22. The means for supplying power to the puncture device 21 is not limited to the battery 22, and some other power supply means may be employed instead. The booster circuit 23 does not necessarily have to be provided to the puncture device 21.

The output 23b of the booster circuit 23 is connected to the positive side 24a of a capacitor 24 having an electrostatic capacity of 300 μf, the anode 26a of a flash lamp 26 (an example of a light source), one of the selection terminals 29a of a switch 29 formed from a semiconductor (an example of the switching circuit), the input 30a of a trigger circuit 30 that generates a high voltage of 2 to 10 kV, and the input 31a of a voltage detection circuit 31 (an example of a laser detector for detecting the stopping of a laser beam) that detects voltage applied to the flash lamp 26. A sensor that detects the stopping of a laser beam 25a directly may be used in place of the voltage detection circuit 31

The negative side 24b of the capacitor 24 is connected to the shared terminal 32c of a switch 32 formed from a semiconductor (an example of a switching circuit). One of the selection terminal 32a of this switch 32 is grounded. The other selection terminal 32b of the switch 32 is connected to the other selection terminal 29b of the switch 29. The shared terminal 29c of the switch 29 is connected to the positive side 33a of a capacitor 33 having an electrostatic capacity of 300 μF. The negative side 33b of the capacitor 33 is grounded.

The cathode side 26c of the flash lamp 26 is connected to the first end 34a of a chop switch 34. The second end 34b of the chop switch 34 is grounded. This chop switch 34 stops the emission of light by the flash lamp 26, so a high-voltage, large-current, high-response IGBT (insulated gate bipolar transistor) is used. An IGBT is used for both the switches 29 and 32. The switches 29 and 32 can also be mechanical switches, as long as they are able to withstand a high voltage of about 500 V and a large current of about 500 A.

A puncture button 36, a power button 37, and the output 31b of the voltage detection circuit 31 are connected to the input of a controller 35. The output of the controller 35 is connected to the control terminal 23c of the booster circuit 23 and the control terminal 30c of the trigger circuit 30.

A laser puncture unit 25 has the flash lamp 26 in which xenon gas is sealed, a laser rod 27 disposed near and in parallel with the flash lamp 26, and a lens barrel that converges the light outputted from the flash lamp 26. This laser rod 27 is made of Er:YAG. A total reflection mirror 28a is mounted at one end of the laser rod 27, and a partial transmission mirror 28b with a transmissivity of about 5% is mounted to the other end. When the flash lamp 26 is excited, it emits a laser beam with a wavelength of 2.94 μm. A converging lens 28c is mounted along the optical axis of the laser beam. The converging lens 28c is designed so that the laser beam which is emitted to the surface of skin will have a spot diameter of from 0.1 to 0.5 mm.

Although not depicted in the drawings, the output of the controller 35 is connected to the control terminals of the switches 29 and 32 and the chop switch 34, and controls the switches 29, 32, and 34.

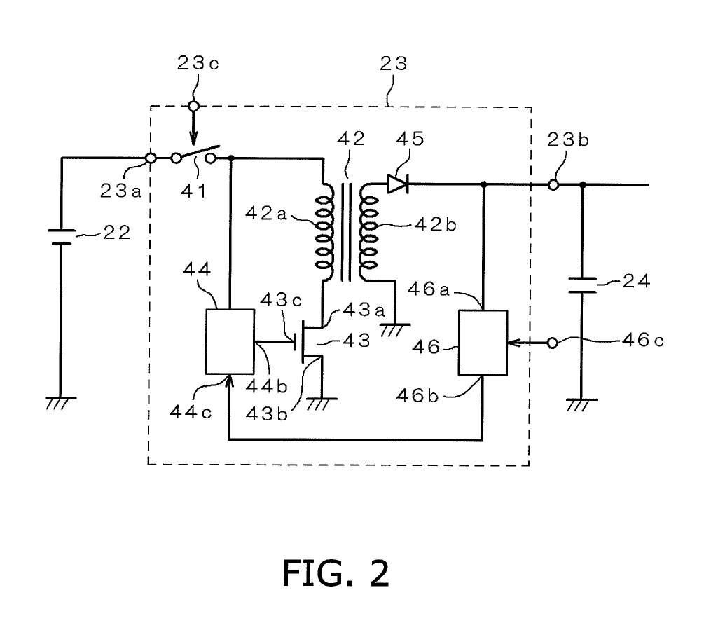

FIG. 2 is a circuit diagram of the booster circuit 23 used in the laser puncture device 21. In FIG. 2, the output of the battery 22 is connected via the input 23a of the booster circuit 23 to one end of an electronic switch 41. The other end of the electronic switch 41 is connected to one end of a primary winding 42a of a transformer 42. The electronic switch 41 is controlled by being connected to the control terminal 23c.

The other end of the primary winding 42a of the transformer 42 is connected to the drain 43a of a field effect transistor 43. The source 43b of the transistor 43 is grounded. The gate 43c of the transistor 43 is connected to the output 44b of an oscillation circuit 44 that oscillates at a frequency of about 100 kHz.

One end of a secondary winding 42b of the transformer 42 is connected to the anode side of a rectifying diode 45. The other end of the secondary winding 42b of the transformer 42 is grounded. The cathode side of the rectifying diode 45 is connected to the output 23b of the booster circuit 23 and to the input 46a of a voltage setting component 46. The output 46b of the voltage setting component 46 is connected to the control terminal 44c of the oscillation circuit 44. The output 23b controls the oscillation pulse width so that the voltage is held steady.

A terminal 46c for setting the voltage from the outside is provided to the voltage setting component 46, and the voltage of the output 23b can be set by controlling this terminal 46c. This means that it is possible to adjust the puncture depth by varying the voltage with which the capacitors 24 and 33 are charged.

The operation of the booster circuit 23 will now be described. The on/off switching of the field effect transistor 43 at high frequency is controlled by the output of the oscillation circuit 44. This switching generates a high voltage on the secondary winding 42b side of the transformer 42. This high voltage is rectified by the diode 45 and supplied to the capacitors 24 and 33, so that the capacitors 24 and 33 are charged.

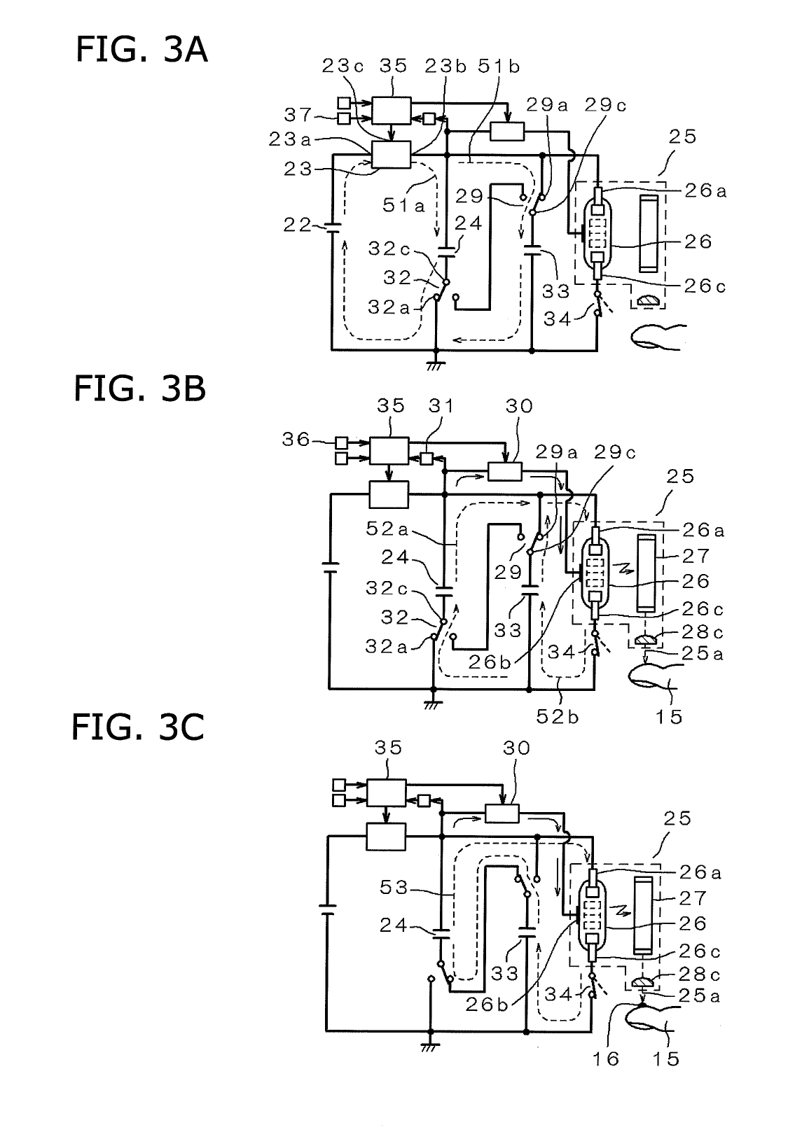

Next, the operation of the puncture device 21 will be described through reference to FIGS. 3A to 3C. FIG. 3A is a block diagram during charging. In FIG. 3A, when the power button 37 is pressed, the controller 35 switches the switch 29 to the selection terminal 29a side, and switches the switch 32 to the selection terminal 32a side. The chop switch 34 is also switched on. At the same time, the booster circuit 23 boosts the 3.7 V voltage of the battery, and the capacitor 24 and the capacitor 33 are each charged to 300 V as indicated by the dotted lines 51a and 51b, respectively. Since the capacitors 24 and 33 here are connected to each other in parallel, the combined capacity is 600 μf.

When the puncture button 36 is pressed in this state, the controller 35 issues a command to apply a trigger voltage of 2 to 10 kV stored in the trigger circuit 30 to a trigger electrode 26b of the flash lamp 26. When the trigger voltage is applied to the trigger electrode 26b, the flash lamp 26 emits light. This excites the laser rod 27, and the laser beam 25a is emitted from the laser rod 27. This laser beam 25a performs a first puncture of the skin 15 through the lens 28c.

After the flash lamp 26 has started emitting light, the controller 35 switches off the chop switch 34 and stops the emission of the flash lamp 26 once the voltage of the anode 26a of the flash lamp 26 reaches 200 V (the voltage at which the emission of the laser beam 25a stops), or after a specific length of time has elapsed (after 200 μs in this embodiment), depending on the output from the voltage detection circuit 31. This allows the emission of light from the flash lamp 26 that does not contribute to laser output to be stopped, which affords higher energy efficiency.

After this, the switch 29 is switched to the other selection terminal 29b, and the switch 32 is also switched to the other selection terminal 32b. Thus, the capacitor 24 and the capacitor 33 are connected in series. The combined capacity of the serial capacitor obtained by this serial connection is 150 μF. Also, the voltage at the two ends of the serial capacitor is two times 200 V (400 V). In this state, the capacitor capacity is smaller than during the first time light was emitted, so the emission time of the flash lamp 26 is shorter, but the optical intensity generated by the flash lamp 26 is larger. This makes it possible to excite the laser rod 27 again.

At a command from the controller 35, the chop switch 34 is switched on and the trigger voltage stored in the trigger circuit 30 is again applied to the trigger electrode 26b of the flash lamp 26. When the trigger voltage is applied to the trigger electrode 26b, the flash lamp 26 emits light. The laser rod 27 that has been excited by the light emitted by the flash lamp 26 emits the laser beam 25a. This laser beam 25a is directed at the skin 15 through the lens 28c, and the second puncture is performed. Consequently, puncture to a greater depth is possible without increasing the puncture surface area on the surface of the skin 15. Thus, puncture can be performed down to the dermis where the veins are found, so blood can seep out from the smallest possible puncture diameter.

After a specific time has passed since the controller 35 has issued a command for the output of trigger voltage to the trigger circuit 30, or after it has been detected that the voltage applied to the capacitors is under 200 V, the switch 29 is switched to the selection terminal 29a. The controller 35 also switches the switch 32 to the selection terminal 32a. This connects the capacitor 24 and the capacitor 33 to each other in parallel. Therefore, the voltage of the parallel capacitor constituted by the capacitors 24 and 33 is one half of 200 V (100 V), which is a safe level.

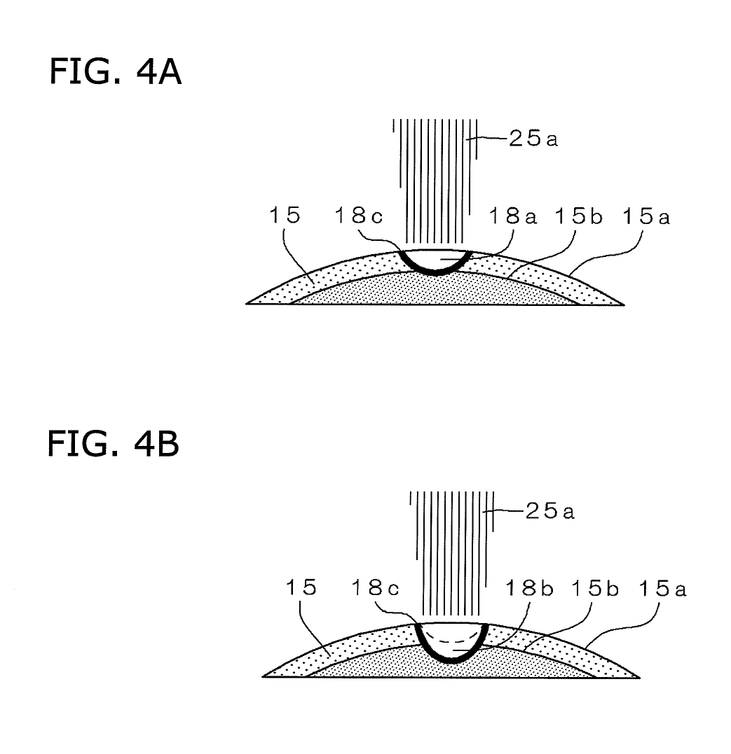

FIGS. 4A and 4B are cross sections of the puncture state of the skin 15 produced by the laser beam 25a. FIG. 4A shows the puncture state after the first puncture, while FIG. 4B shows the puncture state after the second puncture. In the first puncture, the puncture is performed at a laser output that is lower than the output at which blood would seep out from the skin in a single puncture as in the past. Here, because the puncture hole 18a is shallow, no blood comes out, but at the same time, because the puncture diameter is small, the patient experiences less pain. Then, the second puncture is performed at a laser output that is lower than the first laser output. Here, because the puncture goes deeper, blood does come out from the skin 15, but the puncture diameter can be made smaller than when the puncture is made to the same depth all at once. Thus, the pain the patient feels is reduced. Furthermore, since a single laser irradiation takes less time, the heat conduction region 18c is smaller and there tends to be less of a hemostatic effect due to cauterizing, so the blood flows out better.

In other words, compared to when the puncture is made with a single laser irradiation, the volume of the puncture hole 18a is smaller even though the depth of the puncture hole 18a is the same. Furthermore, since the heat conduction region 18c is also smaller, the patient suffers less pain, and the amount of blood collected can also be increased.

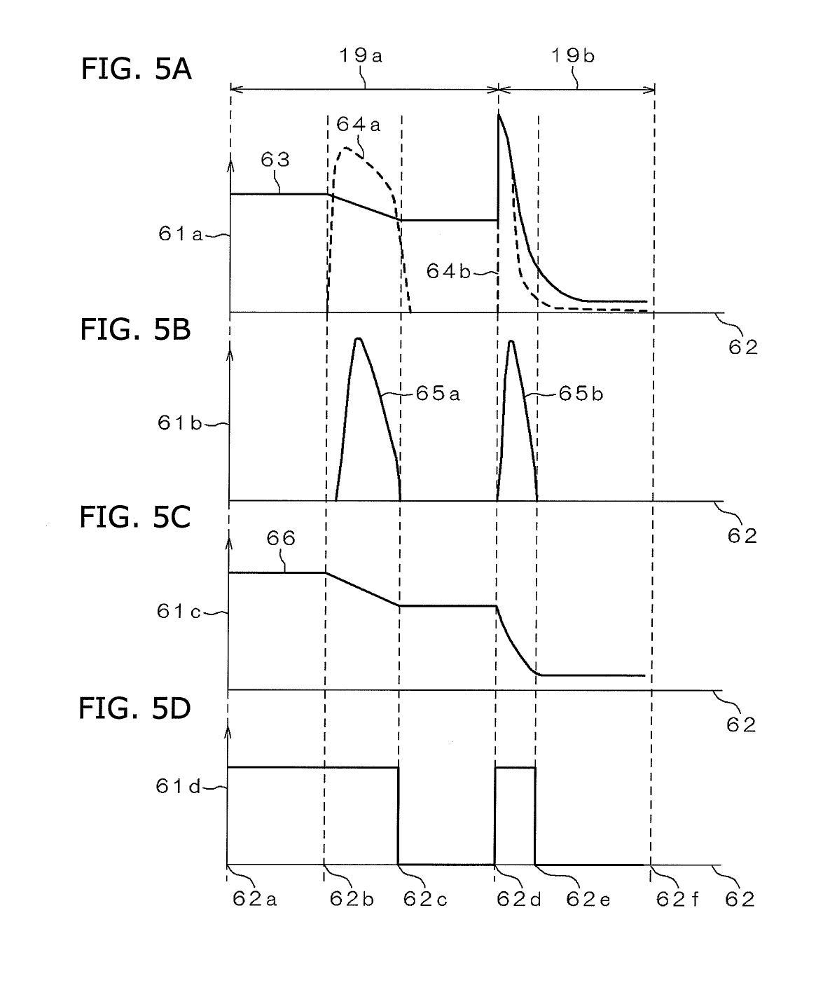

FIGS. 5A to 5C are timing charts of the puncture operation. In FIG. 5A, the vertical axis 61a is the voltage applied to the flash lamp 26, and is the level of the optical intensity of the flash lamp 26. The horizontal axis 62 is the time (sec). The vertical axis 61b hereinafter is the output level of the laser beam 25a, and the vertical axis 61c is the energy level at which the capacitors 24 and 33 are charged. The vertical axis 61d is the operating level of the chop switch 34 that controls whether the flash lamp 26 is on or off. 19a is the period of time the capacitors 24 and 33 are connected in parallel, and 19b is the period of time the capacitors 24 and 33 are connected in series.

In FIG. 5A, the solid line is the voltage 63 applied to the anode 26a of the flash lamp 26, while the dotted line is the intensity of the light emitted from the flash lamp 26 and corresponding to the voltage 63. Of this optical intensity, 64a is the intensity of the light emitted during the first puncture, while 64b is the intensity of the light emitted during the second puncture.

The voltage 63 rises when the power button 37 is pressed, and at point 62a, charging of the serial capacitors that are connected in series is completed. When the puncture button 36 is pressed at point 62b, the flash lamp 26 lights up for the first puncture. As light is emitted from the flash lamp 26, the power with which the capacitors are charged is consumed, and the voltage 63 drops. This drop in the voltage 63 is accompanied by a reduction in the optical intensity 64a as well.

Somewhat after the lighting of the flash lamp 26, the laser beam 25a indicated by the change curve 65a in FIG. 5B is emitted. This change curve 65a changes according to changes in the optical intensity 64a of the flash lamp 26, and when the optical intensity 64a drops below a certain level, the emission of the laser beam 25a is stopped by crossing the excitation threshold of the laser rod 27. In this embodiment, the voltage of the capacitor at this point is 200 V.

At the point 62c at which this voltage of 200 V is detected by the voltage detection circuit 31, or after a specific time has elapsed, the chop switch 34 is switched off. Since the chop switch 34 is switched off, the flash lamp 26 goes out and the optical intensity 64a drops to “0.”

Then, for the second puncture, the controller 35 switches the switch 29 to the other selection terminal 29b side at the point 62d, and switches the switch 32 to the other selection terminal 32b side. As a result, the voltage 63 at the ends of the serial capacitors increases to two times 200 V (400 V). Then, when the chop switch 34 is turned on and a trigger signal is inputted, the flash lamp 26 goes on. As the flash lamp 26 emits light, the voltage with which the capacitors are charged is consumed, and the voltage 63 drops.

As the voltage 63 drops, the optical intensity 64b is also reduced. Corresponding to this optical intensity 64b, the laser beam 25a decreases as indicated by the change curve 65b. When the voltage 63 decreases to under 200 V and the excitation threshold of the laser rod 27 is crossed, the emission of the laser beam 25a stops. At the point 62e when the voltage 63 drops below 200 V, the chop switch 34 shown in FIG. 5D is switched off and the flash lamp 26 goes out.

66 in FIG. 5C is the electrical energy with which the capacitors 24 and 33 are charged in a series of operations. At the point 62a when the charging is complete, the energy with which the capacitors 24 and 33 are initially charged is 27 J, as shown below.

$$\begin{matrix}{E}_{3} = {C}_{2} {V}_{3}^{2} / 2 \\ = ( 600 µ F \times {300}^{2} ) / 2 \\ = 27 ( J )\end{matrix} \\ ( Where {C}_{2} = 600 µ F , and {V}_{3} = 300 V . ) \qquad ( 3 )$$

This 27 J of energy is maintained until the point 62b when the puncture button 36 is pressed. At the point 62c when the puncture button 36 has been pressed and the first puncture is finished, the energy is 12 J, as shown below.

$$\begin{matrix}{E}_{4} = {C}_{2} {V}_{4}^{2} / 2 \\ = ( 600 µ F \times {300}^{2} ) / 2 \\ = 27 ( J )\end{matrix} \\ ( Where {V}_{4} = 200 V . ) \qquad ( 4 )$$

This 12 J of energy is maintained until the point 62d when the second puncture begins. At the point 62e when the second puncture is finished, the energy is 3 J, as shown below.

$$\begin{matrix}{E}_{5} = {C}_{3} {V}_{4}^{2} / 2 \\ = ( 150 µ F \times {200}^{2} ) / 2 \\ = 3 ( J )\end{matrix} \\ ( Where {C}_{3} = 150 µ F . ) \qquad ( 5 )$$

Therefore, the energy used in the first puncture, as shown below, is the energy resulting from subtracting the 12 J of energy after the first puncture from the 27 J of energy at the initial charging, the value of which is 15 J.

$$\begin{matrix}{E}_{6} = {E}_{3} - {E}_{4} \\ = 27 - 12 \\ = 15 ( J )\end{matrix} \qquad ( 6 )$$

Also, the energy used in the second puncture, as shown below, is the energy resulting from subtracting the 3 J of energy after the second puncture from the 12 J of energy after the first puncture, the value of which is 9 J.

$$\begin{matrix}{E}_{7} = {E}_{4} - {E}_{5} \\ = 12 - 3 \\ = 9 ( J )\end{matrix} \qquad ( 7 )$$

Therefore, the energy used in puncture, as shown below, is the sum of the 15 J of energy used in the first puncture and the 9 J of energy used in the second puncture, the value of which is 24 J.

$$\begin{matrix}{E}_{8} = {E}_{6} + {E}_{7} \\ = 15 + 9 \\ = 24 ( J )\end{matrix} \qquad ( 8 )$$

This means that 24 J of energy is used in puncture, which is close to 90% of the 27 J of energy with which the serially connected capacitors 24 and 33 were initially charged, as shown in Formula 3, so there was a pronounced improvement in energy efficiency as compared to a conventional puncture device in which only about 40% of the energy was used in puncture.

In this embodiment, the second puncture is performed by forming a serial capacitor by connecting the capacitors 24 and 33 in series, and thereby converting to the energy required for puncture. Consequently, there is no need for the battery 22 to supply the second puncture energy, so there is a major energy saving overall.

Furthermore, the energy with which the parallel connected capacitors 24 and 33 were charged is used for the first puncture, and in the second puncture the capacitors 24 and 33 are connected in series. Also, after the second puncture, the serially connected capacitors 24 and 33 are returned to parallel connection for the sake of safety. Therefore, it is important for the capacitors 24 and 33 to have equal electrostatic capacities.

Also, the two capacitors 24 and 33 were used in this embodiment, but the same effect as above can also be obtained with a constitution in which three or more capacitors are switched between parallel and serial connection depending on the threshold of laser excitation.

### Embodiment 2

A blood testing device 101 will be described in this embodiment, in which the laser puncture device 21 described in Embodiment 1 above is used. The components having the same function as in Embodiment 1 above will be numbered the same and not described again.

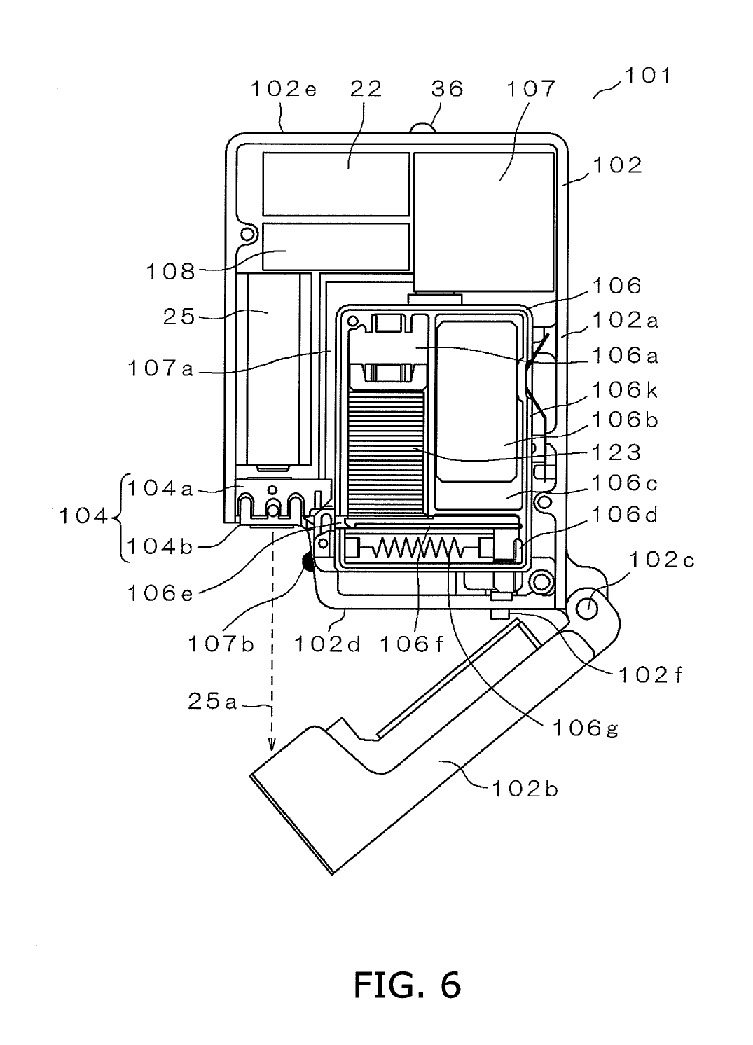

FIG. 6 is a cross section of the blood testing device 101 in this embodiment. In FIG. 6, 102 is a housing with a cuboid shape, and is molded from plastic.

This housing 102 has a main body 102a and a cover 102b rotatably provided to the main body 102a via a fulcrum 102c. Whether the cover 102b is open or closed is detected by an opening sensor 102f mounted on the lower side 102d of the main body 102a. The cover 102b can be latched at a first opening angle of approximately 30 degrees or a second opening angle of approximately 90 degrees.

A puncture component 104 in which blood sensors (hereinafter referred to as “sensors”) 123 are inserted and supported is provided to the corner of the lower side 102d of the main body 102a. The laser puncture unit 25 (the one used in Embodiment 1 above) is mounted opposite this puncture component 104. A sensor cartridge 106 is removably inserted adjacent and parallel to the puncture component 104 and the laser puncture unit 25. The sensor cartridge 106 is inserted or removed by putting the cover 102b in its second opening angle. Puncture with the laser puncture unit 25 is performed by putting the cover 102b in its first opening angle. Therefore, the laser beam 25a will not accidentally leak to the outside, which is safer.

An electrical circuit 108 is provided above the laser puncture unit 25. The battery 22 is removably installed between the 108 and the upper side 102e of the housing 102. A vacuum means 107 is provided above the sensor cartridge 106. This vacuum means 107 is linked to the puncture component 104 via a vacuum passage 107a.

The various components will now be described in detail.

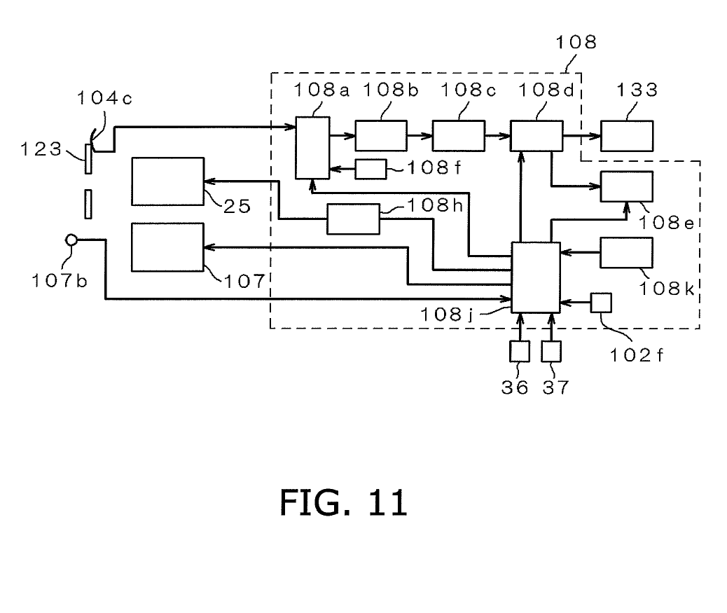

The electrical circuit 108 is supplied with power from the battery 22. The output of this electrical circuit 108 is connected to a display component 133 (see FIG. 11). The electrical circuit 108 measures the blood glucose level of the blood 16 on the basis of a signal from the sensors 123, and displays this level on the display component 133.

106 is a sensor cartridge. This sensor cartridge 106 is molded from plastic and is substantially cuboid in shape. A sensor holding chamber 106a, a desiccant holding chamber 106c, and a conveyance means 106d are provided in the case 106k of this sensor cartridge 106. The sensors 123 are stacked and held in the sensor holding chamber 106a. The desiccant holding chamber 106c is provided in parallel with the sensor holding chamber 106a, and holds a desiccant 106b. The conveyance means 106d is provided below the desiccant holding chamber 106c, and conveys the sensors 123.

The conveyance means 106d has a slide plate 106f and a spring 106g that biases this slide plate 106f. The slide plate 106f conveys the lowest sensor 123 out of the sensors 123 stacked in the sensor holding chamber 106a, through an outlet 106e to the puncture component 104. The slide plate 106f returns to its home position (initial state) under the force of the spring 106g once the conveyance of the sensors 123 is complete.

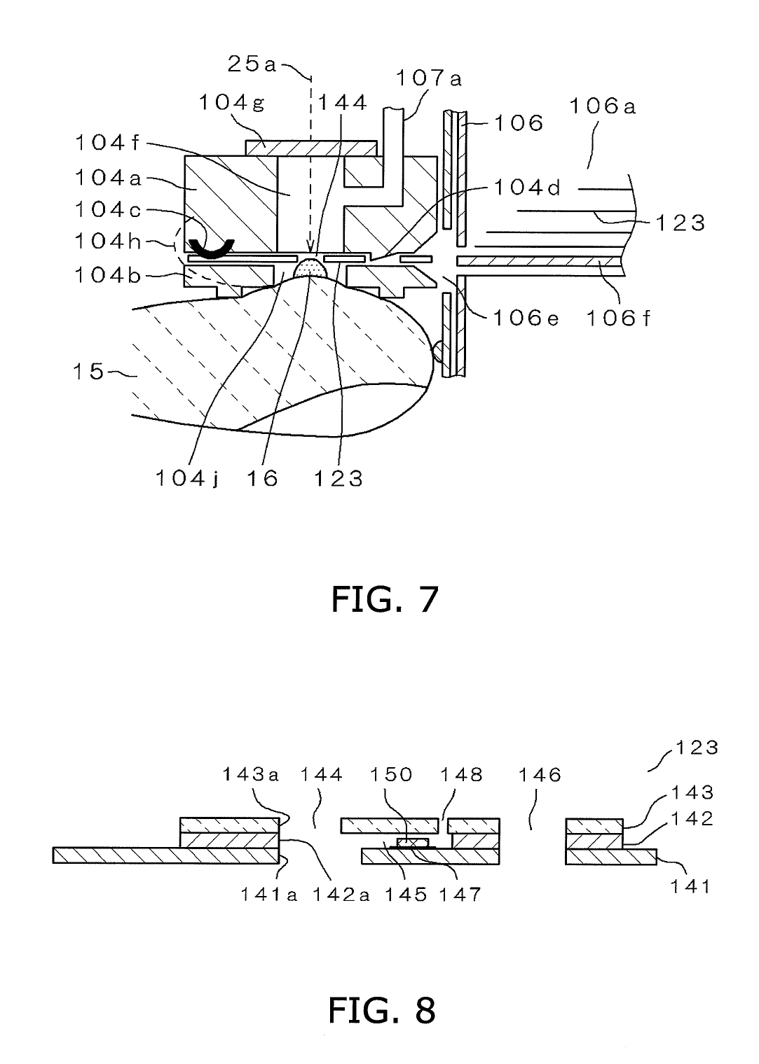

FIG. 7 is a cross section of the puncture component 104 and its surroundings.

The puncture component 104 has an upper holder 104a and a lower holder 104b. The lowest sensor 123 out of the sensors 123 stacked in the sensor holding chamber 106a is conveyed to this puncture component 104. The lowest sensor 123 is sandwiched and fixed between the upper holder 104a and the lower holder 104b.

One end of the puncture component 104 is linked to the outlet 106e of the sensor cartridge 106. A connector 104c is mounted at the other end of the upper holder 104a of the puncture component 104. This connector 104c is provided at a location where it comes into contact with connecting electrodes 151a to 155a and 157a (see FIG. 9) of the sensors 123 set in the puncture component 104.

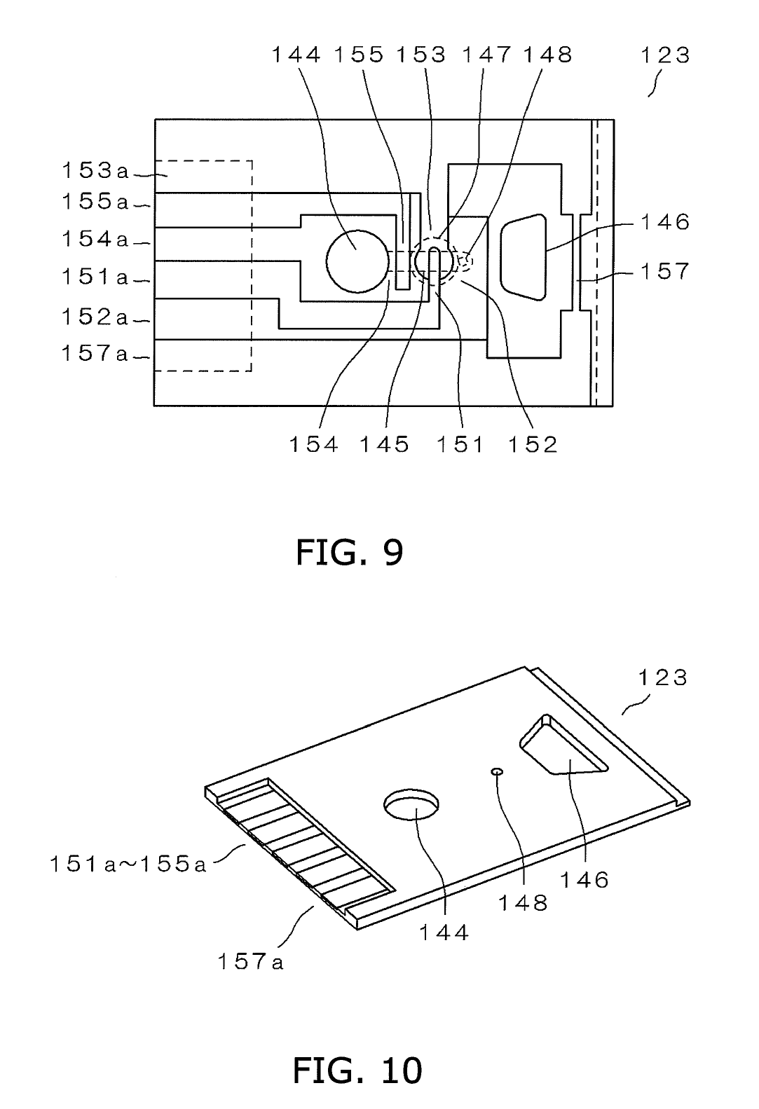

A positioning bump 104d that mates with a positioning hole 146 (see FIGS. 8 to 10) of the sensors 123 is formed on the lower face of the upper holder 104a. This positioning bump 104d mates with the positioning hole 146 of the sensors 123, and positions the sensors 123 at the specified location inside the puncture component 104.

A through-hole 104f is provided in the approximate center of the upper holder 104a. The upper face of this through-hole 104f is sealed with a transparent (transmits the laser beam 25a) film 104g. The vacuum passage 107a from the vacuum means 107 is linked to this through-hole 104f, allowing negative pressure to be applied inside the through-hole 104f.

The lower holder 104b is biased upward by a leaf spring 104h. A through-hole 104j is formed in the approximate center of the lower holder 104b. This through-hole 104j, a reservoir 144 (see FIGS. 8 to 10) of the sensor 123, and the through-hole 104f formed in the upper holder 104a are all formed in a straight line. The laser beam 25a passes through the interior of these to puncture the skin 15. When the skin 15 is punctured, the blood 16 that seeps out of the skin 15 is captured in the reservoir 144 of the sensor 123.

107b is a skin detecting sensor provided to the main body 102a that forms the side face of the puncture component 104. This skin detecting sensor 107b detects contact with the skin 15.

FIG. 8 is a cross section of one of the sensors 123 that are stacked and held in the sensor cartridge 106. This sensor 123 has a substrate 141, a spacer 142 that is stuck to the upper face of the substrate 141, and a cover 143 that is stuck to the upper face of the spacer 142.

144 is a reservoir for the blood 16 (see FIGS. 1 and 7). This reservoir 144 is formed such that a substrate hole 141a formed in the approximate center of the substrate 141, a spacer hole 142a formed in the spacer 142 corresponding to this substrate hole 141a, and a cover hole 143a formed in the cover 143 corresponding to the substrate hole 141a communicate with each other. 146 is a positioning hole for determining the position where the sensors 123 are installed in the puncture component 104, and is provided passing through the sensors 123. This positioning hole 146 mates with the positioning bump 104d formed on the upper holder 104a (see FIG. 7). This positions the sensors 123 in the puncture component 104.

145 is a supply path for the blood 16, and is linked at one end to the reservoir 144. The supply path 145 guides the blood 16 collected in the reservoir 144 to a detector 147 quickly by capillary action. The other end of the supply path 145 is linked to an air hole 148. The volume of the reservoir 144 is 0.904 μL, and the volume of the supply path 145 is 0.144 μL. Thus, the test requires only a small amount of blood 16, which is easier on the patient.

150 is a reagent that is placed on the detector 147. This reagent 150 is formed by adding and dissolving PQQ-GDH (0.1 to 5.0 U/sensor), potassium ferricyanide (10 to 200 mM), maltitol (1 to 50 mM), and taurine (20 to 200 mM) in a 0.01 to 2.0 wt % CMC aqueous solution to prepare a reagent aqueous solution, putting a drop of this on detecting electrodes 151 and 153 (see FIG. 9) formed on the substrate 141, and drying. If this reagent 150 gets damp, its performance deteriorates. To prevent this from happening, the desiccant 106b is held inside the sensor cartridge 106.

Here, the detecting electrodes 151 to 155 (see FIG. 9), connecting electrodes 151a to 155a that are taken off from these detecting electrodes 151 to 155, and an identifying electrode 157a are formed integrally on the upper face of the substrate 141. These are formed by forming an electroconductive layer by sputtering or vapor deposition, using a material such as gold, platinum, or palladium, and subjecting this to laser working.

The material of the substrate 141, the spacer 142, and the cover 143 is polyethylene terephthalate (PET). Using the same material for all three reduces the costs entailed.

FIG. 9 is a see-through plan view of a sensor 123, at one end of which are formed the connecting electrodes 151a to 155a and the identifying electrode 157a. An identification component 157 formed from a conductor pattern is formed between the connecting electrode 153a and the identifying electrode 157a.

144 is a reservoir for the blood 16, and is formed in the approximate center of the sensor 123. The supply path 145 is connected at one end to the reservoir 144, and is provided facing the detecting electrode 152. The other end of the supply path 145 is linked to the air hole 148. The detecting electrode 154 connected to the connecting electrode 154a, the detecting electrode 155 connected to the connecting electrode 155a, the detecting electrode 154 reconnected to the connecting electrode 154a, the detecting electrode 153 connected to the connecting electrode 153a, the detecting electrode 151 connected to the connecting electrode 151a, the detecting electrode 153 reconnected to the connecting electrode 153a, and the detecting electrode 152 connected to the connecting electrode 152a are provided along the supply path 145 in that order, starting from the reservoir 144 side. The reagent 150 (see FIG. 8) is placed on the detecting electrodes 151 and 153.

The sensor 123 can determine whether or not it has been mounted to the puncture component 104 from whether or not there is electrical conduction between the connecting electrode 153a and the identifying electrode 157a. Specifically, when this sensor 123 is conveyed to the puncture component 104, if electrical conduction is detected between the connecting electrode 153a and the identifying electrode 157a, it is determined that the sensor 123 has been properly mounted to the puncture component 104. If there is no electrical conduction between these two, then it is determined that the sensor 123 has not been mounted to the puncture component 104. In this case, a warning can be displayed on the display component 133 of the blood testing device 101 (see FIG. 11).

It is also possible to store information about a calibration curve that is used, or to store manufacturing information, by varying the electrical resistance of the identification component 157. Therefore, this information can be used to conduct a more precise blood test.

FIG. 10 is an oblique view of a sensor 123. This sensor 123 is formed as a rectangular plate. The reservoir 144 is formed in the approximate center of this plate. The connecting electrodes 151a to 155a and the identifying electrode 157a are formed at one end. The positioning hole 146 is formed near the other end. The positioning hole 146 has a trapezoidal shape that tapers to the reservoir 144 side. The air hole 148 is formed between the positioning hole 146 and the reservoir 144.

FIG. 11 is a block diagram of the electrical circuit 108 and its surroundings. In FIG. 11, the identifying electrode 157a and the connecting electrodes 151a to 155a of the sensor 123 (see FIG. 9) are each connected to a switching circuit 108a via the connector 104c provided to the upper holder 104a. The output of the switching circuit 108a is connected to the input of a current/voltage converter 108b. The output thereof is connected to the input of a computer 108d via an analog/digital converter (hereinafter referred to as an A/D converter) 108c. The output of this computer 108d is connected to a transmitter 108e and the display component 133 formed from liquid crystal. The switching circuit 108a is connected to a reference voltage supply 108f. This reference voltage supply 108f may be a ground potential.

108j is a controller. The controller 108j includes the controller 35 that controls the laser puncture unit 25 and was described in Embodiment 1 above. The output of this controller 108j is connected to a high voltage generating circuit 108h connected to the laser puncture unit 25, a control terminal of the switching circuit 108a, the computer 108d, the transmitter 108e, and the vacuum means 107. Also, the puncture button 36 for emitting the laser beam 25a, the power button 37, the opening sensor 102f, the skin detecting sensor 107b, and a timer 108k are connected to the input of the controller 108j.

The high voltage generating circuit 108h is mainly an electrical circuit having the capacitors 24 and 33, as described in Embodiment 1 above.

The operation for measuring a blood glucose level will now be described.

First, the puncture button 36 is pressed to puncture the skin 15 with the laser puncture unit 25. This puncture comprises the first and second punctures described in Embodiment 1 above. The properties of the blood 16 that seeps out after the second puncture are measured. In the measurement of the properties of the blood 16, the switching circuit 108a is switched so that the detecting electrode 151 (see FIG. 9) is connected to the current/voltage converter 108b. The detecting electrode 152, which is used to detect the inflow of the blood 16, is connected to the reference voltage supply 108f. A specific voltage is then applied between the detecting electrode 151 and the detecting electrode 152. In this state, if the blood 16 flows in, current flows between the detecting electrodes 151 and 152. This current is converted into voltage by the current/voltage converter 108b. The voltage value thereof is converted into a digital value by the A/D converter 108c, which is outputted toward the computer 108d. The computer 108d detects that enough blood 16 has flowed in on the basis of this digital value. At this point the operation of the vacuum means 107 is shut off.

Next, the measurement of glucose, which is a blood component, is performed.

To measure the glucose content, first, the switching circuit 108a is switched at a command from the controller 108j, and the detecting electrode 151, which serves as a working electrode for measuring the glucose content, is connected to the current/voltage converter 108b. The detecting electrode 153, which serves as a counter electrode for measuring the glucose content, is connected to the reference voltage supply 108f.

The current/voltage converter 108b and the reference voltage supply 108f are left off while the glucose in the blood and its redox enzyme are reacted for a specific length of time. After a specific length of time has elapsed (1 to 10 seconds), a specific voltage (0.2 to 0.5 V) is applied between the detecting electrodes 151 and 153 at a command from the controller 108j. This results in the flow of current between the detecting electrodes 151 and 153. This current is converted into voltage by the current/voltage converter 108b. The voltage value thereof is converted into a digital value by the A/D converter 108c, which is outputted toward the computer 108d. The computer 108d converts this digital value into a glucose content.

After the glucose content has been measured, the measurement of the Hct level is performed. The Hct level is measured as follows.

First, the switching circuit 108a is switched under a command from the controller 108j. Then, the detecting electrode 155, which serves as a working electrode for measuring the Hct level, is connected to the current/voltage converter 108b. The detecting electrode 151, which serves as a counter electrode for measuring the Hct level, is connected to reference voltage supply 108f.

Next, a specific voltage (2 to 3 V) is applied between the detecting electrodes 151 and 155 from the current/voltage converter 108b and the reference voltage supply 108f under a command from the controller 108j. The current that flows between the detecting electrodes 151 and 155 is converted into voltage by the current/voltage converter 108b. The voltage value thereof is converted into a digital value by the A/D converter 108c, which is outputted toward the computer 108d. The computer 108d converts this digital value into an Hct level.

Using the Hct level and glucose content obtained by these measurements, a predetermined calibration curve or calibration curve table is referred to, and the glucose content is corrected with the Hct level. This corrected result is then displayed on the display component 133. The corrected result is sent from the transmitter 108e to an injection device that injects insulin.

In this embodiment, the measurement of glucose was used as an example, but the present invention is not limited to this.

For instance, the reagent 150 of the sensors 123 can be replaced, and the present invention can be applied to the measurement of lactic acid or cholesterol in the blood, instead of measuring glucose.

The high voltage generating circuit 108h that applies high voltage to the laser puncture unit 25 and is a part of the electrical circuit 108 was substantially described in Embodiment 1 above, so it will not be described again.

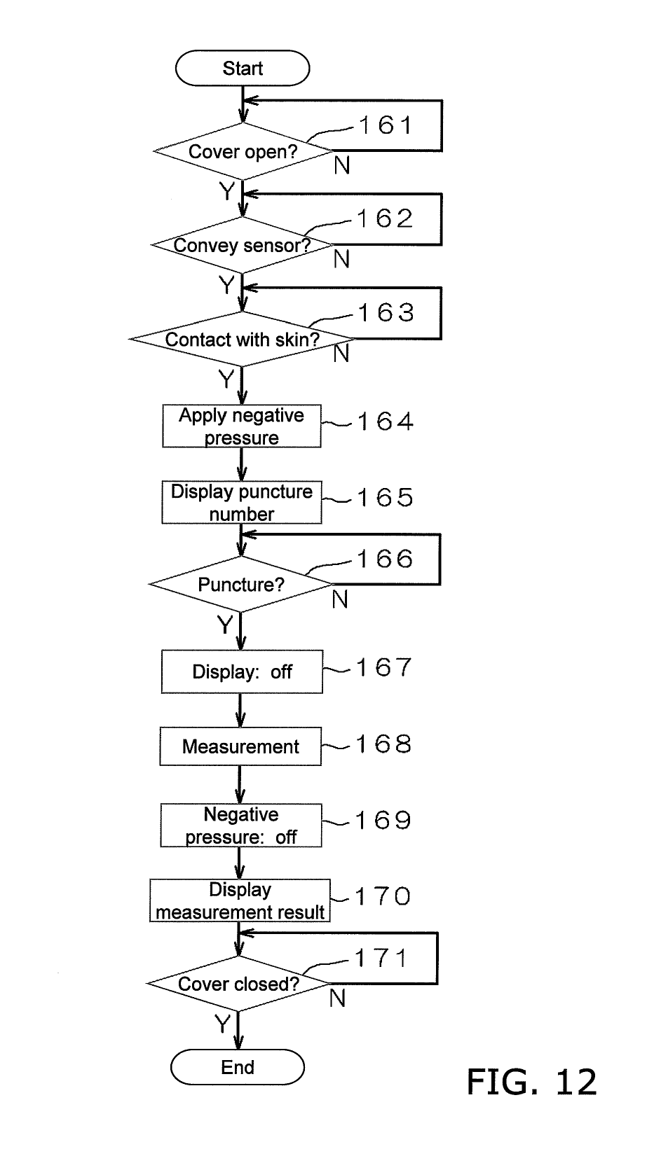

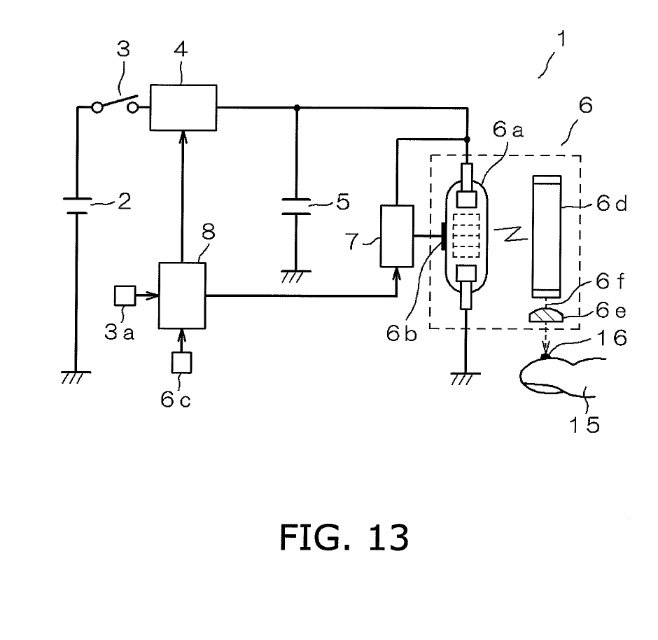

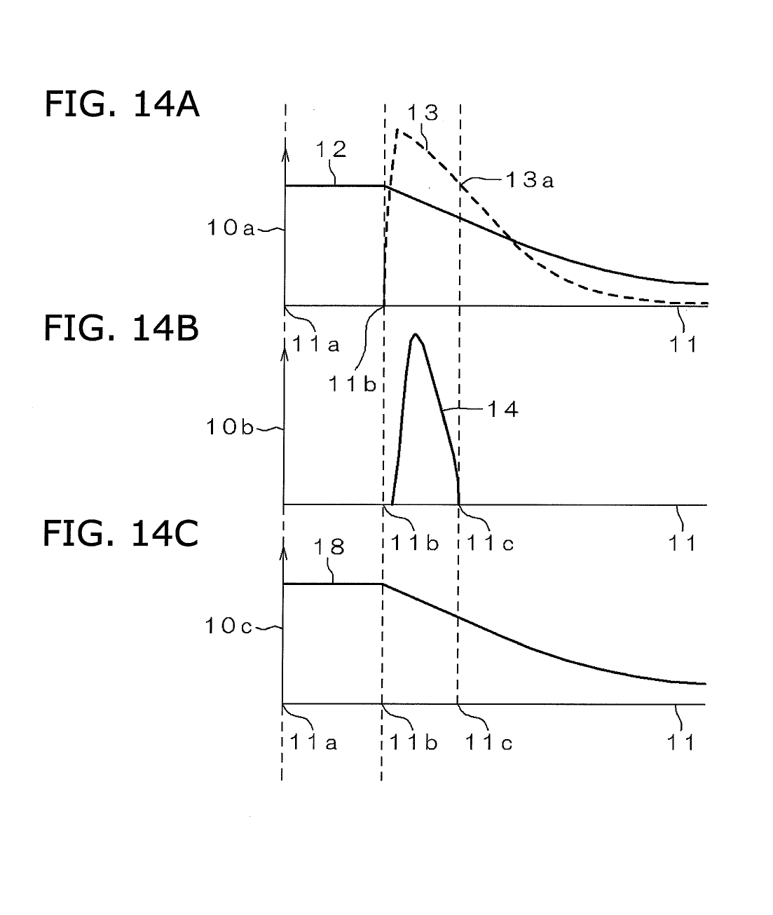

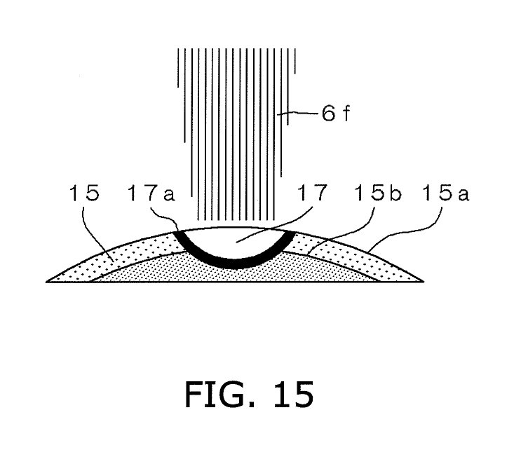

Next, a test method using the blood testing device 101 will be described through reference to FIG. 12.

First, in step 161, the cover 102b of the blood testing device 101 is opened. The open state of the cover 102b is detected by the opening sensor 102f.

Next, the flow moves to step 162, in which the slide plate 106f is moved in the direction of the outlet 106e of the sensor cartridge 106.

This allows the lowest sensor 123 out of those stacked and held to be conveyed to the puncture component 104. The conveyance of the sensor 123 can be started automatically by a signal from the opening sensor 102f. The end of conveyance is confirmed by detecting conduction between the identifying electrode 157a and the connecting electrode 153a (see FIG. 9) of the sensor 123. After this, the slide plate 106f returns to standby mode under the force of the spring 106g.

In step 162, after the sensor 123 has been conveyed, the system checks for conduction between the connecting electrode 153a and the identifying electrode 157a to ascertain whether or not a sensor 123 is present in the sensor cartridge 106. If there is no sensor 123 in the sensor cartridge 106, a display to this effect is given on the display component 133. If the display indicates that there is no sensor 123, then the sensor cartridge 106 is replaced with a new one.

Next, the flow moves to step 163. In step 163 the blood testing device 101 is brought into contact with the skin 15 of the patient. Contact with the skin 15 can be detected by detecting the output of the skin detecting sensor 107b. If contact with the skin 15 is confirmed, the flow moves to step 164, the vacuum means 107 is actuated, and negative pressure is applied to the puncture component 104. As shown in FIG. 7, this negative pressure is applied to the skin 15 via the vacuum passage 107a, the through-hole 104f, the reservoir 144, and the through-hole 104j. Applying negative pressure lifts up the skin 15.

Once a change in current accompanying the operation of the vacuum means 107 is detected, or once a predetermined amount of time has elapsed on the timer 108k, it is determined that the skin 15 has been sufficiently lifted up by the negative pressure inside the lower holder 104b, and the flow moves to step 165.

In step 165, the display component 133 shows a display to the effect that puncture is possible. The flow then moves to step 166, and the system awaits the pressing of the puncture button 36.

When the puncture button 36 is pressed, as described in Embodiment 1 above, the second puncture is performed at the same place. This puncture can also be performed automatically. If it is done automatically, the patient is preferably notified of the puncture timing, either audibly or by a display on the display component 133.

When the puncture button 36 is pressed, the flow moves to step 167. In step 167, the display on the display component 133 performed in step 165 is stopped. The flow then moves to step 168.

In step 168, the blood 16 that seeped out after the second puncture of the skin 15 is captured in the reservoir 144 of the sensor 123. The blood 16 captured in the reservoir 144 is moved quickly (at a set flow rate) by the capillary action of the supply path 145 to the detector 147. The blood glucose level of the blood 16 is then measured.

If the blood glucose level is measured in step 168, the flow moves to step 169 and the vacuum means 107 is turned off. The flow then moves to step 170.

In step 170 the measured blood glucose level is displayed on the display component 133. The vacuum means 107 may instead be switched off at the point when the blood 16 reaches the detecting electrode 152. This ends the measurement of the blood 16, and the flow moves to step 171.

In step 171 the cover 102b of the blood testing device 101 is closed. The closed state of the cover 102b is detected by the opening sensor 102f.

As described above, the puncture of step 166 is divided up into two punctures at the same place on the skin surface as discussed in Embodiment 1 above, so a blood testing device can be obtained with which the diameter of the puncture wound is smaller and which inflicts less pain on the patient. Also, since the remaining energy left over from the first puncture is utilized in the second puncture, so energy can be used more efficiently, and puncture can be performed at a high energy efficiency.

### INDUSTRIAL APPLICABILITY

Using the laser puncture device pertaining to the present invention reduces power consumption, which is particularly useful when applied to a portable blood testing device or the like.

1. A puncture device, comprising:
a laser rod configured to output a laser beam for puncturing a finger;
a flash lamp configured to excite the laser rod;
a plurality of capacitors configured to apply voltage to the flash lamp;
a power supply configured to charge the capacitors;
a switching component configured to switch the connection of the plurality of capacitors between parallel connection and serial connection; and
a controller configured to control the emission timing of the flash lamp and control the switching component so that a first puncture is performed by connecting the plurality of capacitors in parallel, and a second puncture is performed by connecting the plurality of capacitors in series.

2. The puncture device according to claim 1,
further comprising a chop switch configured to connect the flash lamp and the capacitors.

3. The puncture device according to claim 1,
further comprising a converging lens configured to converge the laser beam outputted from the laser rod.

4. The puncture device according to claim 1,
wherein the switching component includes a transistor.

5. The puncture device according to claim 1,
wherein the power supply is a battery.

6. A method for controlling a puncture device that comprises a laser rod configured to output a laser beam for puncturing a finger, a flash lamp configured to excite the laser rod, a plurality of capacitors configured to apply voltage to the flash lamp, a power supply configured to charge the capacitors, a switching component configured to switch the connection of the plurality of capacitors between parallel connection and serial connection, and a controller configured to control the emission timing of the flash lamp and control the switching component so that a first puncture is performed by connecting the plurality of capacitors in parallel, and a second puncture is performed by connecting the plurality of capacitors in series,
the method comprising the steps of:
connecting a plurality of capacitors in parallel;
charging the plurality of capacitors with electrical power;
activating the flash lamp to output a laser beam;
switching the plurality of capacitors to a serial connection; and
activating the flash lamp to output a laser beam.

7. The method for controlling a puncture device according to claim 6,
further comprising the step of connecting the plurality of capacitors in parallel after the step of activating the flash lamp to output a laser beam.

8. A method for controlling a puncture device that comprises a laser rod configured to output a laser beam for puncturing a finger, a flash lamp configured to excite the laser rod, a plurality of capacitors configured to apply voltage to the flash lamp, a power supply configured to charge the capacitors, a switching component configured to switch the connection of the plurality of capacitors between parallel connection and serial connection, a controller configured to control the emission timing of the flash lamp and control the switching component so that a first puncture is performed by connecting the plurality of capacitors in parallel, and a second puncture is performed by connecting the plurality of capacitors in series, and a chop switch that connects the flash lamp and the capacitors,
the method comprising the steps of:
connecting a plurality of capacitors in parallel;
charging the plurality of capacitors with electrical power;
activating the flash lamp to output a laser beam;
switching off the chop switch and switching off the emission of the flash lamp after a specific length of time has elapsed;
switching the plurality of capacitors to a serial connection;
switching on the chop switch after a specific length of time has elapsed; and
activating the flash lamp to output a laser beam.

9. The method for controlling a puncture device according to claim 8,
further comprising the step of switching the plurality of capacitors to parallel connection after the step of activating the flash lamp to output a laser beam.

10. A puncture device, comprising:
a laser output component configured to output a laser beam for puncturing a finger;
an excitation component configured to excite the laser output component;
a plurality of capacitors configured to apply voltage to the excitation component;
a switching component configured to switch the connection of the plurality of capacitors between parallel connection and serial connection; and
a controller configured to control the switching component so that puncture is performed a plurality of times while the connection state of the plurality of capacitors is switched between parallel connection and serial connection.
